(19) United States
(12) Patent Application Publication
(10) Pub. No.: US 2025/0339232 A1
(43) Pub. Date: Nov. 6, 2025

(54) Medical Imaging System with Optical Identifier

(71) Applicant: Bard Access Systems, Inc., Salt Lake City, UT (US)

(72) Inventors: Matthew J. Prince, Herriman, UT (US); Shayne Messerly, Kaysville, UT (US)

(21) Appl. No.: 18/655,066

(22) Filed: May 3, 2024

(51) Int. Cl. A61B 90/00; A61B 90/98

(52) U.S. Cl. CPC A61B 90/37; A61B 90/98; A61B 2090/376; A61B 2090/378

(57) ABSTRACT

A medical imaging system including an optical identifier camera for imaging a medical device, a medical device packaging, a patient, or a technician. The system further includes an imaging probe for imaging of a target location, and a console with optical identifier logic to extract identifier markers or characteristics from the image of the medical device, packaging, patient or technician. A parameter logic determines the medical imaging parameters based on the identifiers or characteristics, and a medical imaging logic captures medical images of the medical device. The system enables efficient and accurate imaging of medical devices on the specific patient, by a specific technician, enhancing diagnostic capabilities and treatment planning in medical parameters.

### BACKGROUND

Previous approaches to imaging medical devices have typically involved a technician modifying the parameters of an imaging system by hand in order to optimize visualization for the specific medical device in use. These techniques often rely on the skill and experience of the technician, or trial and error, in order to find the preferred parameters. However, this approach leads to differing results between different technicians and different imaging events.

To aid the technician in providing standardized imaging parameters, some medical devices are provided with computer readable medium such as magnetic stripes, radio-frequency identification (RFID) chips, or the like, disposed on the medical device, the packaging, or both. The standardized imaging parameters can be stored on the computer readable medium which can then be scanned by the imaging system and the information and parameters for the specific medical device can be uploaded to the imaging system. However, the costs involved with adding and programming the computer readable medium to each medical device and packaging makes this system cost prohibitive for smaller and cheaper medical devices.

Disclosed herein are optical medical device identification systems and methods directed to address the foregoing.

### SUMMARY

In some aspects, the techniques described herein relate to a medical imaging and optical identifier system including, an optical identifier camera configured to generate an identifier image of a medical device or a packaging of the medical device, an imaging probe configured to send and receive a signal for providing a medical image of a target location, a console configured to display one or both of the identifier image and the medical image, and one or more logic modules stored within a non-transitory storage medium, the one or more logic modules, when executed by one or more processors, perform operations including, parsing the identifier image to extract one or more identifier markers, analyzing the one or more identifier markers to determine an identity of the medical device, and modifying a parameter of the medical image based on the identity of the medical device.

In some aspects, the techniques described herein relate to a medical imaging and optical identifier system, wherein parsing the identifier image to extract the one or more identifier markers further includes parsing the identifier image into pixels and assigning a numerical value to each pixel and analyzing the numerical values to detect one or more edges within the identifier image.

In some aspects, the techniques described herein relate to a medical imaging and optical identifier system, wherein parsing the identifier image to extract the one or more identifier markers further includes analyzing the one or more edges detected within the identifier image to detect one or more boundaries within the identifier image.

In some aspects, the techniques described herein relate to a medical imaging and optical identifier system, wherein the one or more identifier markers includes one or more of a boundary of the medical device, a boundary of the packaging, a boundary of a portion of the medical device, a boundary of a portion of the packaging.

In some aspects, the techniques described herein relate to a medical imaging and optical identifier system, wherein analyzing the one or more identifier markers to determine the identity of the medical device further includes comparing one or more of a shape and a dimension of the one or more boundaries within the identifier image with a labeled dataset including a plurality of boundaries and associated medical device identities.

In some aspects, the techniques described herein relate to a medical imaging and optical identifier system, wherein parsing the identifier image to extract the one or more identifier markers further includes parsing the identifier image into pixels an assigning a numerical value to each pixel and analyzing the numerical values to detect one or more colors within the identifier image.

In some aspects, the techniques described herein relate to a medical imaging and optical identifier system, wherein analyzing the one or more identifier markers to determine the identity of the medical device further includes comparing the one or more colors within the identifier image with a labeled dataset including a plurality of colors and associated medical device identities.

In some aspects, the techniques described herein relate to a medical imaging and optical identifier system, wherein parsing the identifier image to extract the one or more identifier markers further includes analyzing the one or more edges detected within the identifier image to detect one or more alphanumeric symbols within the identifier image.

In some aspects, the techniques described herein relate to a medical imaging and optical identifier system, wherein analyzing the one or more identifier markers to determine the identity of the medical device further includes running a text recognition sub-logic module including comparing the one or more alphanumeric symbols within the identifier image with a labeled dataset including a plurality of alphanumeric symbols associated with medical device identities.

In some aspects, the techniques described herein relate to a medical imaging and optical identifier system, wherein the identity of the medical device includes one or more of a model, batch number, serial number, product code, product name, and product description.

In some aspects, the techniques described herein relate to a medical imaging and optical identifier system, wherein determining the identity of the medical device further includes training a machine learning model using a predetermined labeled dataset having a plurality of images, each image of the plurality of images includes one or more labeled identifier markers.

In some aspects, the techniques described herein relate to a medical imaging and optical identifier system, wherein determining the identity of the medical device further includes providing a GUI to request an input confirming the identity of the medical device is correct.

In some aspects, the techniques described herein relate to a medical imaging and optical identifier system, wherein the GUI provides a shortlist of one or more items, each item of the one or more items includes a medical device identity and is ordered in descending order of probability of matching the identity of the medical device.

In some aspects, the techniques described herein relate to a medical imaging and optical identifier system, wherein the imaging probe is an ultrasound probe configured to emit ultrasound signals and detect reflected ultrasound signals, and wherein the one or more logic modules is configured to retrieve information from the ultrasound probe to provide the medical image.

In some aspects, the techniques described herein relate to a medical imaging and optical identifier system, wherein the imaging probe is an electromagnetic probe configured to emit electromagnetic signals and detect reflected electromagnetic signals, and wherein the one or more logic modules is configured to retrieve information from the electromagnetic probe to provide the medical image.

In some aspects, the techniques described herein relate to a medical imaging and optical identifier system, wherein the imaging probe is a fluoroscopic imaging probe configured to emit radiation and detect reflected radiation signals, and wherein the one or more logic modules is configured to retrieve information from the fluoroscopic imaging probe to provide the medical image.

In some aspects, the techniques described herein relate to a medical imaging and optical identifier system, wherein modifying the parameter of the medical image further includes modifying one or more of frequency, gain, depth, focus, dynamic range, persistence, one or more filters, zoom, pan, time gain compensation (“TGC”), color doppler and power doppler.

In some aspects, the techniques described herein relate to a medical imaging and optical identifier system, wherein modifying the parameter of the medical image further includes modifying one or more icons superimposed on the medical image.

In some aspects, the techniques described herein relate to a medical imaging and optical identifier system, wherein the medical image of the medical device is an image of the medical device disposed subcutaneously.

In some aspects, the techniques described herein relate to a medical imaging and optical identifier system, further including the optical identifier camera configured to generate an identifier image of a patient or a technician, and the one or more logic modules configured to analyze the one or more identifier markers to determine a characteristic or an identity of the patient or the technician.

In some aspects, the techniques described herein relate to a medical imaging and optical identifier system, wherein the characteristic of the patient or the technician includes age, gender, weight, height.

In some aspects, the techniques described herein relate to a medical imaging and optical identifier system, wherein determine the identity the patient or the technician includes running one of a facial recognition or a fingerprint recognition analysis to extract the one or more identifier markers and compare the one or more identifier markers with a medical records database.

In some aspects, the techniques described herein relate to a method of placing a medical device including, generating an identifier image of one or both of the medical device and a packaging of the medical device using an optical identifier camera, parsing the identifier image to extract one or more identifier markers, analyzing the one or more identifier markers to determine an identity of the medical device, querying the medical device identity with one or more relational databases to retrieve a predetermined medical imaging parameter, imaging a target location using a probe of a medical imaging system to generate a medical image, modifying a medical imaging parameter of the medical imaging system to the predetermined medical imaging parameter, and placing the medical device at the target location.

In some aspects, the techniques described herein relate to a method, wherein parsing the identifier image to extract the one or more identifier markers further includes parsing the identifier image into pixels and assigning a numerical value to each pixel and analyzing the numerical values to detect one or more edges within the identifier image.

In some aspects, the techniques described herein relate to a method, wherein parsing the identifier image to extract the one or more identifier markers further includes analyzing the one or more edges detected within the identifier image to detect one or more boundaries within the identifier image.

In some aspects, the techniques described herein relate to a method, wherein the one or more identifier markers includes one or more of a boundary of the medical device, a boundary of the packaging, a boundary of a portion of the medical device, a boundary of a portion of the packaging.

In some aspects, the techniques described herein relate to a method, wherein analyzing the one or more identifier markers to determine the identity of the medical device further includes comparing one or more of a shape and a dimension of the one or more boundaries within the identifier image with a labeled dataset including a plurality of boundaries and associated medical device identities.

In some aspects, the techniques described herein relate to a method, wherein parsing the identifier image to extract the one or more identifier markers further includes parsing the identifier image into pixels an assigning a numerical value to each pixel and analyzing the numerical values to detect one or more colors within the identifier image.

In some aspects, the techniques described herein relate to a method, wherein analyzing the one or more identifier markers to determine the identity of the medical device further includes comparing the one or more colors within the identifier image with a labeled dataset including a plurality of colors and associated medical device identities.

In some aspects, the techniques described herein relate to a method, wherein parsing the identifier image to extract the one or more identifier markers further includes analyzing the one or more edges detected within the identifier image to detect one or more alphanumeric symbols within the identifier image.

In some aspects, the techniques described herein relate to a method, wherein analyzing the one or more identifier markers to determine the identity of the medical device further includes running a text recognition sub-logic module including comparing the one or more alphanumeric symbols within the identifier image with a labeled dataset including a plurality of alphanumeric symbols associated with medical device identities.

In some aspects, the techniques described herein relate to a method, wherein the identity of the medical device includes one or more of a model, batch number, serial number, product code, product name, and product description.

In some aspects, the techniques described herein relate to a method, wherein the medical imaging system is one of an ultrasound medical imaging system, electromagnetic medical imaging system, fluoroscopic imaging system, or a magnetic imaging system.

In some aspects, the techniques described herein relate to a method, wherein modifying a parameter of the medical imaging system further includes modifying an icon superimposed on the medical image of the target location.

### BRIEF DESCRIPTION OF DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1 shows a schematic view of a medical imaging system including an optical identifier, in accordance with embodiments disclosed herein.

FIG. 2A shows an exemplary optical identifier image of a first medical device, in accordance with embodiments disclosed herein.

FIG. 2B shows an exemplary optical identifier image of a second medical device, in accordance with embodiments disclosed herein.

FIG. 3A shows an exemplary optical identifier image of a first packaging, in accordance with embodiments disclosed herein.

FIG. 3B shows an exemplary optical identifier image of a second packaging, in accordance with embodiments disclosed herein.

FIG. 4A shows an exemplary optical identifier image of a first medical device disposed within a first packaging, in accordance with embodiments disclosed herein.

FIG. 4B shows an exemplary optical identifier image of a second medical device disposed in a second packaging, in accordance with embodiments disclosed herein.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein. It is understood that the drawings are diagrammatic and schematic representations of exemplary embodiments of the invention and are neither limiting nor necessarily drawn to scale.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “right,” “left,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise. Also, the words “including,” “has,” and “having,” as used herein, including the claims, shall have the same meaning as the word “comprising.”

In the following description, the terms “or” and “and/or” as used herein are to be interpreted as inclusive or meaning any one or any combination. As an example, “A, B or C” or “A, B and/or C” mean “any of the following, A, B, C, A and B, A and C, B and C, A, B and C.” An exception to this definition will occur only when a combination of elements, components, functions, steps or acts are in some way inherently mutually exclusive.

With respect to “proximal,” a “proximal portion” or a “proximal end portion” of, for example, a catheter or system disclosed herein includes a portion of the catheter or system intended to be near or relatively nearer to a clinician when the catheter or system is used on a patient. Likewise, a “proximal length” of, for example, the catheter or system includes a length of the catheter or system intended to be near or relatively nearer to the clinician when the catheter or system is used on the patient. A “proximal end” of, for example, the catheter or system includes an end of the catheter or system intended to be near or relatively nearer to the clinician when the catheter or system is used on the patient. The proximal portion, the proximal end portion, or the proximal length of the catheter or system can include the proximal end of the catheter or system; however, the proximal portion, the proximal end portion, or the proximal length of the catheter or system need not include the proximal end of the catheter or system. That is, unless context suggests otherwise, the proximal portion, the proximal end portion, or the proximal length of the catheter or system is not necessarily a terminal portion or terminal length of the catheter or system.

With respect to “distal,” a “distal portion” or a “distal end portion” of, for example, a catheter or system disclosed herein includes a portion of the catheter or system intended to be near or relatively nearer to a patient when the catheter or system is used on a patient. Likewise, a “distal length” of, for example, the catheter or system includes a length of the catheter or system intended to be near or relatively nearer to the patient when the catheter or system is used on the patient. A “distal end” of, for example, the catheter or system includes an end of the catheter or system intended to be near or relatively nearer to the patient when the catheter or system is used on the patient. The distal portion, the distal end portion, or the distal length of the catheter or system can include the distal end of the catheter or system; however, the distal portion, the distal end portion, or the distal length of the catheter or system need not include the distal end of the catheter or system. That is, unless context suggests otherwise, the distal portion, the distal end portion, or the distal length of the catheter or system is not necessarily a terminal portion or terminal length of the catheter or system.

The term “logic” may be representative of hardware, firmware or software that is configured to perform one or more functions. As hardware, the term logic may refer to or include circuitry having data processing and/or storage functionality. Examples of such circuitry may include, but are not limited or restricted to a hardware processor (e.g., microprocessor, one or more processor cores, a digital signal processor, a programmable gate array, a microcontroller, an application specific integrated circuit “ASIC”, etc.), a semiconductor memory, or combinatorial elements.

Additionally, or in the alternative, the term logic may refer to or include software such as one or more processes, one or more instances, Application Programming Interface(s) (API), subroutine(s), function(s), applet(s), servlet(s), routine(s), source code, object code, shared library/dynamic link library (DLL), or even one or more instructions. This software may be stored in any type of a suitable non-transitory storage medium, or transitory storage medium (e.g., electrical, optical, acoustical or other form of propagated signals such as carrier waves, infrared signals, or digital signals). Examples of a non-transitory storage medium may include, but are not limited or restricted to a programmable circuit; non-persistent storage such as volatile memory (e.g., any type of random access memory “RAM”); or persistent storage such as non-volatile memory (e.g., read-only memory “ROM”, power-backed RAM, flash memory, phase-change memory, etc.), a solid-state drive, hard disk drive, an optical disc drive, or a portable memory device. As firmware, the logic may be stored in persistent storage.

Unless defined otherwise, all technical and scientific terms used herein have the same meaning as commonly understood by those of ordinary skill in the art.

### System Architecture

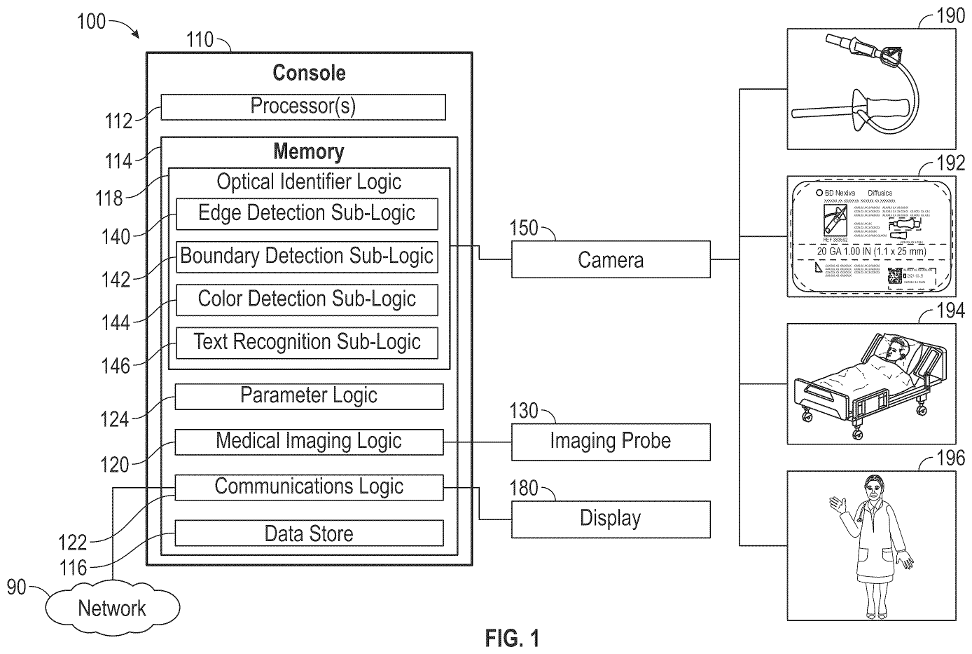

FIG. 1 shows an exemplary medical imaging and optical identifier system (“system”) 100 generally including a console 110 having a display 180, an imaging probe 130, and an optical identifier camera (“camera”) 160. In an embodiment, the console 110 includes one or more processors 112, memory 114, data store 116, and one or more logic engines and/or sub-logic engines, for example, an optical identifier logic 118, a medical imaging logic (“imaging logic”) 120, a parameter logic 124, and a communications logic 122. It will be appreciated that the console 110 can take one of a variety of forms and may include additional components (e.g., power supplies, ports, interfaces, etc.) that are not directed to aspects of the disclosure. The one or more processors 112, with access to the memory 114 (e.g., non-volatile memory or non-transitory, computer-readable medium), are included to control functionality of the console 110 during operation.

In an embodiment, the one or more logic engines may receive and process data, as described herein. The one or more logic engines may be in the form of a software application that is loaded on the console 110 and executable by the one or more processors 112. In other embodiments, the one or more logic engines need not be loaded on the console 110 but may instead execute within a cloud computing environment (which may also be represented by the network 90) such that data from the memory 114 are communicated to the one or more logic engines for processing, for example by way of the communications logic 122. Thus, any of the one or more logic engines represented as being part of the console 110 may include an application programming interface (API) that is configured to transmit and receive data communication messages to and from the one or more logic engines operating in the cloud computing environment, i.e., network 90. In an embodiment, the one or more logic engines may use one or more of predetermined rule sets, machine learning schema, artificial intelligence (“AI”), neural networks, text recognition schema, image recognition schema, or the like to execute the functionality of the console 110 during operation.

In an embodiment, the optical identifier logic 118 can be configured to send and receive signals to/from the optical identifier camera 150 and generate an identifier image 158 of a medical device 190, a packaging 192 of the medical device 190, a patient 194, a technician 196, combinations thereof, or the like. Exemplary medical devices 190 can include peripheral intravenous catheters (“PIVC”), central venous catheter (CVC), peripherally inserted central catheter (PICC), rapidly insertable central catheters (RICC), arterial catheters, pulmonary artery catheter, dialysis catheter, implantable ports, tunneled catheters, combinations thereof, or the like. However, it will be appreciated that these medical devices are exemplary and not considered to be limiting in any way.

Exemplary packaging 192 of the medical device 190 can include boxes, labels, plastic envelopes, film barriers, release liners, shrink wraps and can be formed of paper, cardboard, plastic, polymers, gauze, woven or non-woven materials, organic or synthetic materials, combinations thereof, or the like. In an embodiment, as described in more detail herein, the optical identifier camera 150 can image a patient 194, or a portion thereof, and the optical identifier logic 118 can determine one or more characteristics or an identity of the patient 194. Exemplary characteristics of the patient can include gender, age, weight, height, combinations thereof, or the like. In an embodiment, as described in more detail herein, the optical identifier camera 150 can image a technician 196, or a portion thereof, and the optical identifier logic 118 can determine one or more characteristics, or an identity of the technician 196, as described herein. In an embodiment, the camera 150 can be a still camera that captures “still” images, a video camera, a digital camera, or combinations thereof.

In an embodiment, the optical identifier logic 118 is configured analyze the identifier image 158 of the medical device 190 and/or the packaging 192 and extract one or more identifier markers 160 to determine the identity of the medical device 190 being used. Exemplary identifier markers 160 can include, but not limited to, an outline of the medical device 190 or a portion thereof, a color of the medical device 190 or a portion thereof, a barcode or a “QR” code disposed on one or both of the medical device 190 and the packaging 192, a symbol or combination of symbols disposed on one or both of the medical device 190 and the packaging 192, such as a shape, icon, alphanumeric symbol, combinations thereof, or the like. Exemplary identities of the medical device 190 can include make, model, batch number, serial number, product code, product name, product description, combinations thereof, or the like.

Once the identity of the medical device 190 is determined using the one or more identifier markers 160, the system 100 can use the medical device identity to modify a parameter of the medical imaging logic 120 for providing a medical image of the medical device 190 at a target location. Exemplary target locations can include on or within the body of a patient.

By way of a non-limiting example, the system 100 can generate an identifier image 158 of a medical device 190, such as a PIV catheter. The optical identifier logic 118 can parse the identifier image 158 and extract one or more identifier markers 160, such as an outline of the medical device 190 as a whole, an outline of a portion of the medical device 190 such as a hub, a color of the medical device 190, or a portion thereof, or a serial number written on the medical device 190. The system 100 can then analyze the one or more identifier markers 160 to determine the identity of the medical device 190, for example by running a comparison algorithm against one or more relational databases, by analyzing using a trained machine learning model, combinations thereof, or the like. Optionally, the system can confirm the identity of the medical device 190 by requesting an input from a user. Optionally, once the identity of the medical device 190 is confirmed, the system 100 can retrieve additional information about the medical device 190. Once the identity of the medical device 190 is confirmed, the system 100 can modify a parameter of the medical imaging logic 120 based on the identity of the medical device 190, the practical application of which can include modifying the parameter to a predetermined parameter best suited to image the medical device 190 at the target location. Alternatively, or in addition to, modifying the parameter can include superimposing an icon on the medical image to represent the medical device at the target location. These and other embodiments will be discussed in more detail herein.

In an embodiment, the console 110 further includes a display 180 to show the medical image of the medical device 190 on or within the body of the patient 194, and/or track a location thereof. In an embodiment, the display 180 is formed integrally with the console 110. In an embodiment, the display 180 is a separate, standalone device that is communicatively coupled with the console 110 either wired or wirelessly. Optionally, the display 180 includes a touch screen configured to receive an input from a user. Other user interfaces are also contemplated to fall within the scope of the present invention including keyboards, mouse, trackpads, microphones for voice inputs, gyroscopes, accelerometers, cameras, combinations thereof or the like.

### Identifier Image Processing

In an embodiment, the optical identifier logic 118 identifies and classifies objects, shapes, colors, alphanumeric symbols (e.g., text and/or numbers) from the identifier image 158 generated by the optical identifier camera 150. The optical identifier logic 118 parses the identifier image 158 into pixels, assigns a numerical value that represents color and intensity for each pixel, and analyzes the relative arrangement for each pixel within the identifier image 158.

In an embodiment, the optical identifier logic 118 module includes one or more sub-logic modules configured for image feature extraction, and including but not limited to, edge detection 140, boundary detection 142, color detection 144, text recognition 146, combinations thereof, and the like. Additional sub-logic modules can include texture detection, pattern detection, object recognition, biomarker recognition (gender, age, height, weight), identity recognition (facial recognition, fingerprint recognition), combinations thereof, or the like.

In an embodiment, the optical identifier logic 118 includes an edge detection sub-logic module 140 configured to analyze the numerical value of a first pixel and analyze the numerical values of one or more adjacent pixels and run analyses, statistical analysis, or the like, to determine significant changes in numerical values between adjacent pixels that would indicate significant changes in color or intensity between adjacent pixels that would indicate a depiction of an edge within the identifier image 158. In an embodiment, the edge detection sub-logic module 140 can run one or more layers of analysis with each layer including different groupings of pixels and comparing significant changes between adjacent groupings of pixels. These multi-layer analysis can remove “noise” from the analysis, providing a statistical smoothing to the data and increase the accuracy of the edge detection.

In an embodiment, the optical identifier logic 118 includes a boundary detection sub-logic module 142 configured to detect a continuous outer boundary within the identifier image 158. For example, the boundary detection sub-logic module 142 can be configured to analyze the pixels of the identifier image 158 and/or information received from the edge detection sub-logic module 140 to determine patterns of one or more edges within identifier image 158 and determine combinations of edges that provide a boundary within the identifier image 158. The boundary detection sub-logic module 142 can be configured to determine the shape, dimensions, perimeter distance, and the like, of one or more the boundaries within the identifier image 158.

In an embodiment, the optical identifier logic 118 includes a texture recognition sub-logic module configured to analyze the pixels of the identifier image 158 and/or information received from one or more sub-logic modules to determine patterns of pixel color and intensity that denote different textures within the identifier image 158. Such textures can denote different materials, surface structures, three-dimensional shapes, or the like.

In an embodiment, the optical identifier logic 118 includes an object recognition sub-logic module configured to analyze the pixels of the identifier image 158 and or information generated by one or more sub-logic modules to determine the shape, color, and/or texture of an object within the identifier image 158 and how closely the object resembles one or more objects from a labeled dataset. For example, the object recognition sub-logic module can use one or more statistical analysis, predetermined rulesets and weightings, neural networks, machine learning (M.L.) schema, artificial intelligence (A.I.) to compare an object identified from the identifier image 158, along with associated parameters of shape, dimensions, color, texture, etc., and determine which labeled objects, from the predetermined labeled dataset that it most closely represents. For example, object recognition sub-logic module can access a label dataset of medical devices, stored locally on the console 110, or stored remotely and accessed by way of the network 90 and determine a short list of one or more labeled images of medical devices that most closely represent the object identified in the identifier image 158.

In an embodiment, the optical identifier logic 118 includes a text recognition sub-logic module 146 configured to analyze the pixels of the identifier image 158 and/or information generated by one or more sub-logic modules to determine one or more alphanumeric symbols, or combinations of alphanumeric symbols within the identifier image 158. The text recognition sub-logic module 146 can use one or more statistical analysis, predetermined rulesets and weightings, neural networks, machine learning (M.L.) schema, artificial intelligence (A.I.) to compare the one or more alphanumeric symbols with labeled datasets of medical devices having known text, numbers, serial numbers, batch numbers, combinations thereof, or the like.

In an embodiment, the optical identifier logic 118 includes a color detection sub-logic module 144 configured analyze the pixels within the identifier image 158 and/or information generated by one or more sub-logic modules to quantify a color within the identifier image 158 or within a portion of the identifier image 158, e.g. within a boundary of a particular shape. The color detection sub-logic module 144 can use one or more statistical analysis, predetermined rulesets and weightings, neural networks, machine learning (M.L.) schema, artificial intelligence (A.I.) to compare the one or more colors with predetermined labeled datasets, e.g. datasets of medical devices having specific colors used by different manufacturers to denote different makes, models, or batches of medical devices 190, etc.

### Optical Identifier Markers of Medical Device

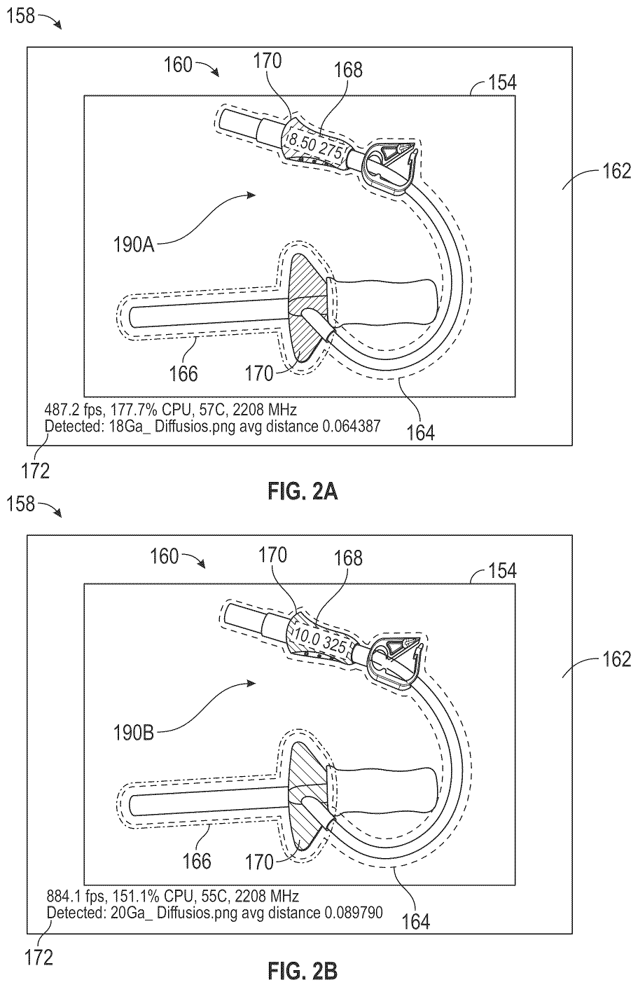

Once the identifier image 158 has been processed, the optical identifier logic 118 can extract one or more identifier markers 160 from the identifier image 158 and use these to determine the identity of the medical device 190. FIGS. 2A-4B show exemplary identifier images 158 captured by the optical identifier camera 150 of the system 100. The identifier images 158 include images of a medical device 190, a packaging 192 of the medical device 190, or combinations thereof, and shows one or more identifier markers 160, associated therewith.

For example, FIG. 2A shows an identifier image 158 of a first medical device 190A, disposed on an identifier platform (“platform”) 162. In an embodiment, the optical identifier logic 118 can analyze the identifier image 158 and extract a first identifier 164, such as an outline, outer perimeter shape, or dimensions (length, width, height, etc.), of the first medical device 190A as a whole. In an embodiment, the optical identifier logic 118 can analyze the identifier image 158 and extract a second identifier 166, such as an outline, outer perimeter shape, or dimensions of a portion of the first medical device 190A, e.g., a hub and needle assembly of the medical device 190, or the like. In an embodiment, the optical identifier logic 118 can analyze the identifier image 158 and extract a third identifier 168, such as a plurality of alphanumeric symbols, text, number codes, serial numbers, batch numbers, product code, product name, product description, or the like, disposed on the first medical device 190A. In an embodiment, the optical identifier logic 118 can employ a text recognition sub-logic module to determine the third identifier 168. In an embodiment, the optical identifier logic 118 can extract a fourth identifier 170, such as a color of the first medical device 190A or a portion thereof such as a color of a catheter hub or connector of the medical device. To note, the top right to bottom left diagonal shading of FIG. 2A is representative of a first color, the top left to bottom right diagonal shading of FIG. 2B is representative of a second color. In an embodiment, the fourth identifier 170 can be defined by the specific hue of the color. For example, the optical identifier logic 118 can determine a specific color code for the color shown on the medical device 190 and use the color code to determine the identity of the medical device. Exemplary color codes include RGB (Red, Green, Blue) code, CMYK (Cyan, Magenta, Yellow, Black) code, HSV (Hue, Saturation, Value) code, Lab (CIELAB) code, HSI (Hue, Saturation, Intensity) code or the like.

The system 100 can use one or more identifier markers 160, e.g., the first identifier 164, the second identifier 166, the third identifier 168, and the fourth identifier 170, to determine the identity of the first medical device 190A. Exemplary medical device identity can include the make, model, batch number, serial number, product code, product name, combinations thereof, or the like. In an embodiment, the system 100 can use the medical device identity to retrieve additional information about the first medical device 190A. Exemplary additional information includes product description, size, dimensions (catheter length, diameter, French size, etc.), and/or material properties of the first medical device 190A. In an embodiment, the identity 172 of the first medical device 190A and/or associated information about the medical device or the identifier image 158 can be displayed on the identifier image 158.

In an embodiment, the system 100 uses the identity of the medical device 190A to query a relational database, data store 116, network 90, cloud-based data store, hospital network, electronic health records, intranet, internet, combinations thereof, or the like to retrieve additional information about the first medical device 190A. Additional information can include one or more of a make, model, batch number, serial number, product code, product name, product description, size, dimensions (catheter length, diameter, French size, etc.), and/or material properties (material used, mechanical properties, etc.) of the first medical device 190A. Exemplary dimensions of the first medical device 190A can include, catheter tube length, inner diameter of one or more lumen, wall thickness, French size, number of lumens, types of connectors, or the like. Exemplary material properties of the first medical device 190A can include, the type of material used (e.g., plastic, polymer, elastomer, metal, alloy, composite, etc.), the mechanical properties of the material (e.g., tensile strength, flexibility, elasticity, etc.) optical imaging opaqueness, acoustic imaging opaqueness, electromagnetic imaging opaqueness, radiographic imaging opaqueness, magnetic imaging opaqueness, combinations thereof, or the like.

FIG. 2B shows an identifier image 158 of one or more identifier markers 160, e.g., a first identifier 164, a second identifier 166, a third identifier 168, and a fourth identifier 170, of a second medical device 190B as identified by the optical identifier logic 118. To note, where the one or more identifier markers 160 provide a different combination of information, i.e., different shape, outline, perimeter, alphanumeric codes, colors, or combinations thereof, the system 100 can determine that the identity of the second medical device 190B is different from the first medical device 190A and display a different identity 172 on the identifier image 158. Further, the system 100 can retrieve additional information relating to the second medical device 190B, as described herein.

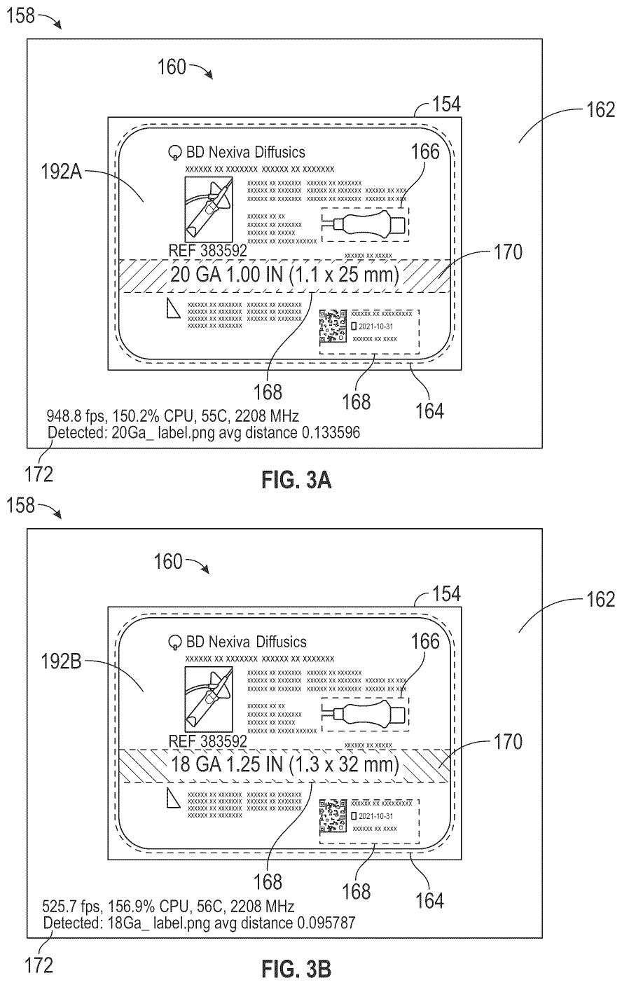

FIG. 3A shows an optical identifier image 158 of a first packaging 192A of the first medical device 190A. In an embodiment, the optical identifier logic 118 can analyze the identifier image 158 and extract a first identifier 164, such as an outline, outer perimeter shape, or dimensions (length, width, height, etc.), of the first packaging 192A as a whole. In an embodiment, the optical identifier logic 118 can analyze the identifier image 158 and extract a second identifier 166, such as an outline, outer perimeter shape, or dimensions of a portion of the first packaging 192A. In an embodiment, the optical identifier logic 118 can extract a third identifier 168, such as a plurality of alphanumeric symbols, text, number codes, serial numbers, batch numbers, product code, product name, product description, bar code, QR code, or the like, disposed on the first packaging 192A. In an embodiment, the optical identifier logic 118 can extract a fourth identifier 170, such as a color of the first packaging 192A or a portion thereof. To note, the top right to bottom left diagonal shading of FIG. 3A is representative of a first color, the top left to bottom right diagonal shading of FIG. 3B is representative of a second color. In an embodiment, the fourth identifier 170 can be defined by the specific hue of the color. For example, the optical identifier logic 118 can determine a specific color code, as described herein, for the color shown on the medical device packaging 192 and use the color code to determine the identity of the medical device associated therewith.

The system 100 can use the one or more identifier markers 160 to determine the identity of the first packaging 192A and the identity of the first medical device 190A associated therewith. The identity of the first packaging 192A and/or the first medical device 190A can include the make, model, batch number, serial number, product code, product name, combinations thereof, or the like, of the first packaging 192A and/or the first medical device 190A associated therewith. In an embodiment, identity 172 of the first medical device 190A and/or information associated therewith, can be displayed on the identifier image 158.

In an embodiment, the system 100 uses the identity of the medical device packaging 192A to query a relational database, data store 116, network 90, cloud-based data store, hospital network, electronic health records, intranet, internet, combinations thereof, or the like to retrieve additional information about the first medical device 190A associated therewith, as described herein.

FIG. 3B shows the one or more identifier markers 160 of a second packaging 192B. To note where the one or more identifier markers 160 provide a different combination of information, i.e., different shape, outline, perimeter, alphanumeric codes, colors, or combinations thereof, the system 100 can determine that this is a different packaging from the first packaging 192A and display a different identity 172 on the identifier image 158. Further, the system 100 can retrieve additional information relating to the second medical device 190B associated with the second packaging 192B.

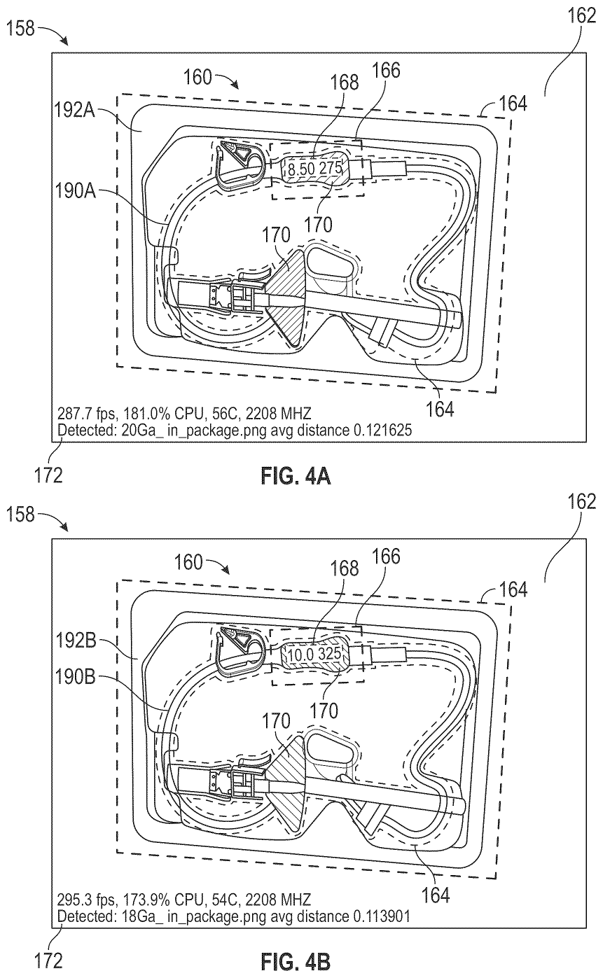

FIG. 4A shows an identifier image 158 of a first medical device 190A disposed within a first packaging 192A and disposed on a platform 162. In an embodiment, at least a portion of the packaging 192 is transparent, or sufficiently translucent, to observe at least a portion of the medical device 190 through the packaging 192. As such, the optical identifier logic 118 can identify one or more identifier markers 160 associated with one or both of the medical device 190 and the packaging 192 without having to remove the medical device 190 from the packaging 192. Advantageously, this maintains the sterility of the medical device 190 up until the moment of use. Alternatively, if the technician 196 is comparing different medical devices for use, the technician 196 can identify the medical device without having to breach the packaging. Further, this mitigates using the incorrect medical device should a different medical device be preferred, or the medical device be packaged incorrectly. Anomalies between the identifier markers 160 of the medical device and the identifier markers 160 of the packaging can be determined by the system 100 and the system 100 can provide a visual, audible, or tactile alert to the technician 196.

In an embodiment, the optical identifier logic 118 can identify a first identifier 164, such as an outline, outer perimeter shape, or dimensions, of the first medical device 190A and/or first medical device packaging 192A, as a whole. In an embodiment, the optical identifier logic 118 can identify a second identifier 166, such as an outline, outer perimeter shape, or dimensions of a portion of one or both of the first medical device 190A and the first medical device packaging 192A. In an embodiment, the optical identifier logic 118 can identify a third identifier 168, such as a plurality of alphanumeric symbols, text, number codes, serial numbers, batch numbers, product code, product name, product description, bar code, QR code, or the like, disposed on one or both of the first medical device 190A and the first medical device packaging 192A.

In an embodiment, the optical identifier logic 118 can identify a fourth identifier 170, such as a color of the first medical device 190A, the first medical device packaging 192A, or a portion thereof. To note, the top right to bottom left diagonal shading of FIG. 4A is representative of a first color, the top left to bottom right diagonal shading of FIG. 3B is representative of a second color. In an embodiment, the fourth identifier 170 can be defined by the specific hue of the color. For example, the optical identifier logic 118 can determine a specific color code, as described herein, for the color shown on the medical device 190 and/or packaging 192 and use the color code to determine the identity of the medical device associated therewith.

The system 100 can use one or more of the identifier markers 160 to determine the identity 172 of the first medical device 190A such as the make, model, product code, product name of the first medical device 190A. In an embodiment, the system 100 uses the identity to query a remote or internal relational database or data store to retrieve additional information about the medical device as described herein. In an embodiment, the identity 172 of the first medical device 190A and information associated therewith, can be displayed on the identifier image 158.

FIG. 4B shows an identifier image 158 including one or more identifier markers 160 of a second medical device 190B disposed within a second medical device packaging 192B. To note where the one or more identifier markers 160 provide a different combination of information, i.e., different shape, outline, perimeter, alphanumeric codes, colors, or combinations thereof, the system 100 can determine that the identity of the second medical device 190B and/or packaging 192B is different from the first medical device 190A and/or packaging 192B and display a different identity 172 and associated information on the identifier image 158.

### Medical Device Identity Analysis

In an embodiment, the system 100 can use one or more identifier markers 160 to differentiate between a medical device, a packaging of the medical device, and a medical device disposed within the packaging. It will be appreciated that the four different identifier markers 160 shown in FIGS. 2A-4B are exemplary and that greater or lesser numbers or combinations of identifier markers 160 are contemplated to fall within the scope of the present invention.

Once the optical identifier logic 118 has analyzed the identifier image 158, extracted the one or more identifier markers 160, the optical identifier logic 118 can then use one or more statistical analysis, predetermined rule sets and weightings, neural networks, machine learning (M.L.) schema, artificial intelligence (A.I.) to determine the identity of the medical device 190.

In an embodiment, the optical identifier logic 118 develops a model to compare the one or more identifier markers 160, extracted from the identifier image 158 with one or more labeled datasets of known medical devices 190, packaging 192, and associated identifiers 160, for example, using one or more predetermined rulesets and weightings. The practical application of which being that the one or more labeled datasets that the identifier markers 160 are being compared against can be maintained and updated with a full range of medical devices.

In an embodiment, the model can be a supervised or unsupervised machine learning model that is trained using one or training datasets of labeled data. The training datasets can include a plurality of images of one or more medical devices 190, packaging 192, etc. The images can be labeled to denote the medical device within the image and to denote the one or more identifier markers 160 associated therewith, along with a medical device identity. Each image within the training dataset can be further labeled to include additional information about the medical device, including make, model, batch number, serial number, dimensions, materials, mechanical properties, combinations thereof, and the like, as described herein. The plurality of images of the training data of the one or more medical devices 190, packaging 192 can include images taken under different lighting conditions, angles, perspectives, shutter speeds, apertures, backgrounds, focus levels, F-stops, combinations thereof, or the like.

The model can be trained using the training datasets before being provided with the identifier image 158 of a medical device was not included as part of the training dataset. The model can then provide a short list of one or more items, with each item of the one or more items representing a known medical device that the model determines most closely represents medical device in the identifier image 158. The practical application of the model being a more flexible image recognition system able to interpret new data and, using previous training data, to provide an identity of the medical device in the identifier image despite the specific image not having been previously labeled.

### Medical Device Identity Confirmation

In an embodiment, once the system 100 had determined a shortlist of one or more proposed medical devices that most closely resembles the identifier image 158, the system 100 can request an input from a user to confirm the identity of the medical device 190 is accurate.

In an embodiment, the system 100 provides the shortlist of one or more items, with each item of the one or more items representing a proposed medical device. In an embodiment, the one or more items can be presented as a table, a list, series of icons, avatars, or thumbnail pictures, combinations thereof, or the like. The system 100 can request input from the user either to select which of the items in the short list is correct, or to confirm if one of the items, i.e., the item that most closely represents the medical device in the image, is correct. If confirmed as correct, the identifier image 158 can be added to the training dataset to augment future model training. If the input from the user confirms that the item is not correct, subsequent items, or groups of items, are provided in an iterative manner until the correct medical device is identified. Where items from the shortlist are confirmed as correct or incorrect, these data can be fed back into the training dataset as labeled data for future training of the model. Where no items on the shortlist are indicated as the correct, the system 100 can then request input from the user, e.g., by way of the display 180, to identify the medical device 190. Once the identity of the medical device 190 has been confirmed, the optical identifier logic 118 can run one or more algorithms to retrieve addition information about the medical device 190 from one or more datasets stored locally on the console 110 or accessed by way of the network 90.

### Medical Imaging Parameters

With continued reference to FIG. 1, in an embodiment, the system 100 further includes an imaging probe 130 configured to send and receive signals, and one or more logic engines, (e.g., imaging logic 120, parameter logic 124) configured to receive information from the imaging probe 130 and derive a medical image of a target location on or within the body of the patient 194, and optionally a medical device disposed at the target location.

In an embodiment, the console 110 includes an ultrasound imaging probe 130 having a transducer configured to emit ultrasound waves and detect reflected ultrasound waves. In an embodiment, the imaging probe 130 is an electromagnetic (“EM”) imaging probe configured to emit electromagnetic waves and detect reflected electromagnetic waves. Exemplary electromagnetic waves can include infrared (“IR”), near infrared (“NIR”), optical, ultraviolet, or combinations thereof. However, it will be appreciated that these are exemplary ranges of EM waves, and greater or lesser electromagnetic wavelengths are also contemplated. In an embodiment, the imaging probe 130 is a fluoroscopic imaging probe configured to emit radioactive waves or particles and detect reflected radioactive waves or particles to determine an image of the medical device 190 disposed on or within the body of the patient 194. In an embodiment, the imaging probe 130 is a magnetic imaging probe configured to detect a static magnetic field from a permanent magnet disposed on the medical device 190. In an embodiment, the imaging probe 130 is configured to use one or more modalities (e.g., optical, acoustic, electromagnetic, radioactive, magnetic, etc.) concurrently. In an embodiment, the imaging logic 120 receives information from the imaging probe 130 and determines a medical image of one or both of a target area and the medical device 190 on or within the body of the patient 194. In an embodiment, the imaging logic 120 receives information from the imaging probe 130 and tracks a location, orientation, and/or trajectory of the medical device 190 relative to the body of the patient 194.

In an embodiment, the parameter logic 124 is configured retrieve the identity of the medical device 190 that is determined by the optical identifier logic 118. The parameter logic 124 then uses the identity of the medical device 190 to query a remote or internal relational database, data store 116, network 90, cloud-based data store, hospital network, electronic health records, intranet, internet, combinations thereof, or the like to retrieve additional information about the medical device 190. Additional information can include, for example, make, model, serial number, batch number, product code, product name, product description, dimensions, catheter French size, material properties, etc., as described herein.

In an embodiment, the parameter logic 124 uses the identity of the medical device 190 to query a remote or internal relational database, data store 116, network 90, etc. to retrieve one or more predetermined imaging parameters for the medical imaging system (e.g., optical, acoustic, electromagnetic, radioactive, magnetic, or combinations thereof) to image the specific medical device 190 while being used on or within the patient 194.

In an embodiment, the parameter logic 124 retrieves the identity of the medical device 190, and any additional information associated therewith, from the optical identifier logic 118 and determines the appropriate imaging parameters for the console 110 to provide a medical image of the medical device 190, or to track the medical device 190 on or within the body. For example, a first medical device 190A may include a first material having a first opaqueness to one of an optical, acoustic, EM, radiation, or magnetic modality and require a first parameter or set of parameters in order to image or track the first medical device 190A relative to the patient 194. Further, a second medical device 190B may include a second material having a second opaqueness to one of an optical, acoustic, EM, radiation, or magnetic modality, different from the first opaqueness and require a second parameter, or set of parameters in order to image or track the first medical device 190A relative to the patient 194.

Exemplary parameters to be modified by the parameter logic 124 include frequency, e.g., ultrasound frequency, EM wavelength, etc., for example, lower frequencies may penetrate deeper, while higher frequencies offer better resolution. Gain, to enhance the visibility of the medical device 190 relative to the surrounding tissues. Depth, focus, dynamic range, persistence to help reduce noise and enhance image clarity, tissue harmonics, image enhancement filters, e.g., to suppress noise and improve image quality, zoom, pan, time gain compensation (“TGC”), e.g., to control the gain at different depths within the image, color doppler and power doppler, to detect movement or flow around the medical device, or combinations thereof. Further parameters can be modified to suit differences in length, size, gauge, French size, dimensions, or materials of the medical device 190.

In an embodiment, the imaging parameters are predetermined and stored locally on data store 116. The parameter logic 124 retrieves the identity of the medical device 190, packaging 192, patient 194, and/or technician 196, and queries a remote or local relational database, data store, or network 90 to retrieve the one or more imaging parameters. In an embodiment, the imaging parameters are stored remotely, e.g., on a hospital network, intranet, internet, cloud storage, etc. and are accessed and retrieved by way of a network 90 and communications logic 122. Advantageously, the image parameters can be updated remotely by the manufacturer, or similar third party. In an embodiment, the imaging logic 120 can retrieve the imaging parameters from the parameter logic and 124 and automatically updates the imaging parameters to optimize for the specific medical device 190, patient 194, and/or technician 196 in use.

In an embodiment, the parameters logic 124 can retrieve the identity of the medical device 190 and modify a parameter of the medical image including providing an icon, image, avatar, or thumbnail superimposed on the medical image. The icon can be representative of the medical device being identified and/or representative of one or more dimensions of the medical device and scaled relative to the target location in the medical image.

For example, the system 100 can superimpose a first circle having a first diameter to represent a catheter diameter of a first medical device 190A. The user can then identify a second medical device 190B using the optical identifier camera 150, which has a larger catheter diameter. The system 100 can identify the second medical device 190B, retrieve the diameter of the catheter and superimpose a second circle having a different diameter on the medical image. While this is a simplified example, the practical application of the system 100 being to allow a user to accurately identify which medical device is being used, retrieve additional information about the medical device, and represent the medical device and additional information at the target location in the medical image without having to remove the medical device from the sterile packaging or place the medical device at the target location.

### Patient or Technician Characteristics and Identity

In an embodiment, the medical imaging and optical identifier system 100 can be used to derive a characteristic or an identity of one or both of the patient 194 and the technician 196 present during the procedure. For example, prior to, or after identifying the medical device 190 and/or packaging 192 to be used in the procedure, the technician 196 can use the optical identifier camera 150 to image the patient 194 as a whole, or a portion thereof, such as a face, hand, fingerprint, combinations thereof, or the like. The optical identifier logic 118 can analyze the identifier image from the camera 150 using one or more sub-logic modules, as described herein, and determine one or more characteristics of the patient 194.

In an embodiment, the optical identifier logic 118 can include a biomarker recognition sub-logic module configured to analyze the identifier image 158 or information generated from one or more sub-logic modules and determine one or more identifier markers that represent a characteristic or an identify of the patient 194 or the technician 196. Exemplary characteristics can include the type of person being imaged, e.g., patient 194 or technician 196, the gender of the person being imaged, age, age range, weight, height, facial landmarks, facial analysis, expression analysis, emotional analysis, pain scale analysis, facial recognition, individual identity, combinations thereof, or the like. In an embodiment, the optical identifier logic 118 can run one or more predetermined rule sets, machine learning schema, artificial intelligence (A.I.), facial recognition, biometric recognition schema, or the like, as described herein, to identify and determine the one or more characteristics of the patient 194. For example, the optical identifier logic 118 can determine that the patient 194 is male or female, pediatric, adult, or geriatric and the system 100 can modify the medical imaging parameters as appropriate, as described herein.

In an embodiment, the optical identifier logic 118 can determine the individual identity of the patient 194 or technician 196 using facial recognition, fingerprint analysis, or the like, and query a local or remote database, e.g., network 90, hospital medical records, etc., to retrieve one or more additional characteristics or additional information regarding the patient 194 or the technician 196 such as age, weight, height, identity, etc. In an embodiment, the optical identifier logic 118 can include an identity recognition sub-logic module configured to analyze the identifier image 158 and/or information generated by one or more sub-logic modules to determine one or more facial landmarks or fingerprint recognition landmarks from the identifier image 158. Exemplary facial recognition landmarks can include an outline of a face or head, outline of the eyes, nose, mouth, ears, eyebrows, and relative locations thereof. Exemplary fingerprint recognition landmarks include patterns of ridges, arches, loops, and wholes. The optical identifier logic 118 can then build a numerical representation of the patient 194 or technician 196 being identified by the relative distances between these landmarks. The optical identifier logic 118 can then run a comparison algorithm to compare these landmarks with datasets of known biomarker or identity recognition markers to determine the identity of the patient 194, technician 196 and associated information. The system 100 can then modify the medical imaging parameters as appropriate, as described herein.

In an embodiment, the optical identifier logic 118 develops a model to compare the one or more biomarkers, characteristics, facial recognition landmarks, etc., extracted from the identifier image 158 with one or more labeled datasets. In an embodiment, the model can be a supervised or unsupervised machine learning model that is trained using one or training datasets of labeled data. The training datasets can include a plurality of images of one or more persons, patients, technicians, hospital staff, etc. The images can be labeled to denote the person within the image and to denote the one or more biomarkers, characteristics, or landmarks associated with the person. For example, the training dataset can include a plurality of images of people of a given age, or age range, height, weight, gender, medical condition etc. and the images can be labeled as such. Where possible, the identity of the person can also be included. Each image within the training dataset can be further labeled to include additional information about the person, as described herein. The plurality of images of the training data can include images taken under different lighting conditions, angles, perspectives, shutter speeds, apertures, backgrounds, focus levels, F-stops, combinations thereof, or the like.

The model can be trained using the training datasets before being provided with the identifier image 158 of a person not included as part of the training dataset. The model can then provide a short list of one or more items, with each item of the one or more items representing a person of known characteristics or identity that the model determines most closely represents person in the identifier image 158. The practical application of the model being a more flexible image recognition system able to interpret new data and, using previous training data, to provide an identity of the medical device in the identifier image despite the specific image not having been previously labeled.

In an embodiment, the medical imaging and optical identifier system 100 can be used to identify the technician 196 operating the medical imaging system 100. The system 100 can then modify the medical imaging parameters to those preferred by the individual technician 196 using the medical imaging system 100, as appropriate, as described herein.

In an exemplary method of use, a medical imaging and optical identifier system 100 is provided, as described herein. Prior to placing the medical device 190, the technician 196 places one or both of the medical device 190 and the packaging 192 in front of the camera 150. In an embodiment, the camera 150 is formed integrally with the console 110 and is directed to a platform 162 on which the medical device 190 and/or packaging 192 can be placed. Advantageously, the platform 162 can be positioned at a predetermined distance from the camera 150 to facilitate focusing and image capture. Optionally, the platform 162 includes one or more guidelines 154 to facilitate aligning the medical device 190 and/or packaging 192 with the camera 150.

In an embodiment, the camera 150 is a separate, standalone device that is communicatively coupled with the console 110, either by wires or wirelessly. Advantageously, the clinician can bring the camera 150 to the medical device 190 and/or packaging 192 to capture an image thereof, facilitating image capture and preventing the medical device 190 and/or packaging 192 from having to be removed from a sterile field. In an embodiment, the technician 196 uses the camera 150 to image the patient 194, or a portion thereof, to determine one or more characteristics of the patient 194, as described herein. In an embodiment, the technician 196 uses the camera 150 to image the technician 196, or a portion thereof, to determine one or more characteristics of the technician 196, as described herein.

The optical identifier logic 118 receives information from the camera 150 and applies one or more of a predetermined rules-based, machine learning, artificial intelligence (A.I.), image recognition, neural networks, text recognition, image recognition schema, facial recognition, biometric marker recognition, or combinations thereof, to identify one or more identifier markers 160 of the medical device 190, the packaging 192, the patient 194, and/or the technician 196 from the image(s) captured by the camera 150. For example, the optical identifier logic 118 can determine one or more of an outline or perimeter of the medical device 190 or a portion of thereof, a barcode or a “QR” code disposed on one or both of the medical device 190 and the packaging 192, a symbol or combination of symbols disposed on one or both of the medical device 190 and the packaging 192, such as a shape, icon, alphanumeric symbol, or combinations thereof, words, number codes, make, model, batch number, serial number, product code, product name, product description written on the medical device 190 or packaging 192, or combinations thereof, or a color of the medical device 190 or a portion thereof. The optical identifier logic 118 uses the one or more identifier markers 160 of the medical device 190 to determine an identity of the medical device 190. For example, the optical identifier logic 118 can determine one or more characteristics of the patient 194 and/or technician 196, or portion thereof, such as age, gender, weight, height, facial recognition, fingerprint recognition, individual identity, combinations thereof, or the like. In an embodiment, the system 100 requests additional confirmation from the technician 196 regarding the information about the medical device 190, packaging 192, patient 194, and/or technician 196, determined by the optical identifier logic 118. Confirmation can be in the form of a visual, audible, or tactile input from the technician 196 to the system 100. Exemplary inputs include a voice command, hand gesture, head nod, tactile input from a physical button, or tactile input on a touchscreen display, combinations thereof, or the like.

Advantageously, the system 100 streamlines and expedites the process of adjusting the imaging parameters to image the specific medical device 190 being used, on the specific patient 194 and/or by the specific technician 196. Advantageously, the system 100 reduces the need for advanced training to adjust the imaging parameters for each medical device 190 being used, allowing a greater range of technicians to use the system 100. Advantageously, the system 100 reduces human error in entering incorrect imaging parameters and providing a sub-optimal imaging of the medical device 190 for the specific patient 194 and mitigates associated errors in placement of the medical device 190. Advantageously, the medical device 190 and/or packaging 192 does not require any RFID chip or similar electronic or computer readable information stored on medical device 190 or packaging 192 itself, simplifying manufacture and reducing associated costs. Advantageously, the system 100 can be used with existing products, third party products, or used retroactively with products already provided, without the need for including system specific computer readable medium with the medical device 190 and/or packaging 192.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. A medical imaging and optical identifier system, comprising:
an optical identifier camera configured to generate an identifier image of a medical device or a packaging of the medical device;
an imaging probe configured to send and receive a signal for providing a medical image of a target location;
a console configured to display one or both of the identifier image and the medical image; and
one or more logic modules stored within a non-transitory storage medium, the one or more logic modules, when executed by one or more processors, perform operations including:
parsing the identifier image to extract one or more identifier markers;
analyzing the one or more identifier markers to determine an identity of the medical device; and
modifying a parameter of the medical image based on the identity of the medical device.

2. The medical imaging and optical identifier system according to claim 1, wherein parsing the identifier image to extract the one or more identifier markers further includes parsing the identifier image into pixels and assigning a numerical value to each pixel and analyzing the numerical values to detect one or more edges within the identifier image.

3. The medical imaging and optical identifier system according to claim 2, wherein parsing the identifier image to extract the one or more identifier markers further includes analyzing the one or more edges detected within the identifier image to detect one or more boundaries within the identifier image.

4. The medical imaging and optical identifier system according to claim 1, wherein the one or more identifier markers includes one or more of a boundary of the medical device, a boundary of the packaging, a boundary of a portion of the medical device, a boundary of a portion of the packaging.

5. The medical imaging and optical identifier system according to claim 3, wherein analyzing the one or more identifier markers to determine the identity of the medical device further includes comparing one or more of a shape and a dimension of the one or more boundaries within the identifier image with a labeled dataset including a plurality of boundaries and associated medical device identities.

6. The medical imaging and optical identifier system according to claim 1, wherein parsing the identifier image to extract the one or more identifier markers further includes parsing the identifier image into pixels an assigning a numerical value to each pixel and analyzing the numerical values to detect one or more colors within the identifier image.

7. The medical imaging and optical identifier system according to claim 6, wherein analyzing the one or more identifier markers to determine the identity of the medical device further includes comparing the one or more colors within the identifier image with a labeled dataset including a plurality of colors and associated medical device identities.

8. The medical imaging and optical identifier system according to claim 2, wherein parsing the identifier image to extract the one or more identifier markers further includes analyzing the one or more edges detected within the identifier image to detect one or more alphanumeric symbols within the identifier image.

9. The medical imaging and optical identifier system according to claim 8, wherein analyzing the one or more identifier markers to determine the identity of the medical device further includes running a text recognition sub-logic module including comparing the one or more alphanumeric symbols within the identifier image with a labeled dataset including a plurality of alphanumeric symbols associated with medical device identities.

10. The medical imaging and optical identifier system according to claim 1, wherein the identity of the medical device includes one or more of a model, batch number, serial number, product code, product name, and product description.

11. The medical imaging and optical identifier system according to claim 1, wherein determining the identity of the medical device further includes training a machine learning model using a predetermined labeled dataset having a plurality of images, each image of the plurality of images includes one or more labeled identifier markers.

12. The medical imaging and optical identifier system according to claim 1, wherein determining the identity of the medical device further includes providing a GUI to request an input confirming the identity of the medical device is correct.

13. The medical imaging and optical identifier system according to claim 12, wherein the GUI provides a shortlist of one or more items, each item of the one or more items includes a medical device identity and is ordered in descending order of probability of matching the identity of the medical device.

14. The medical imaging and optical identifier system according to claim 1, wherein the imaging probe is an ultrasound probe configured to emit ultrasound signals and detect reflected ultrasound signals, and wherein the one or more logic modules is configured to retrieve information from the ultrasound probe to provide the medical image.

15. The medical imaging and optical identifier system according to claim 1, wherein the imaging probe is an electromagnetic probe configured to emit electromagnetic signals and detect reflected electromagnetic signals, and wherein the one or more logic modules is configured to retrieve information from the electromagnetic probe to provide the medical image.

16. The medical imaging and optical identifier system according to claim 1, wherein the imaging probe is a fluoroscopic imaging probe configured to emit radiation and detect reflected radiation signals, and wherein the one or more logic modules is configured to retrieve information from the fluoroscopic imaging probe to provide the medical image.

17. The medical imaging and optical identifier system according to claim 1, wherein modifying the parameter of the medical image further includes modifying one or more of frequency, gain, depth, focus, dynamic range, persistence, one or more filters, zoom, pan, time gain compensation (“TGC”), color doppler and power doppler.

18. The medical imaging and optical identifier system according to claim 1, wherein modifying the parameter of the medical image further includes modifying one or more icons superimposed on the medical image.

19. The medical imaging and optical identifier system according to claim 1, wherein the medical image of the medical device is an image of the medical device disposed subcutaneously.

20. The medical imaging and optical identifier system according to claim 1, further including the optical identifier camera configured to generate an identifier image of a patient or a technician, and the one or more logic modules configured to analyze the one or more identifier markers to determine a characteristic or an identity of the patient or the technician.

21. The medical imaging and optical identifier system according to claim 20, wherein the characteristic of the patient or the technician includes age, gender, weight, height.

22. The medical imaging and optical identifier system according to claim 20, wherein determine the identity the patient or the technician includes running one of a facial recognition or a fingerprint recognition analysis to extract the one or more identifier markers and compare the one or more identifier markers with a medical records database.

23. A method of placing a medical device, comprising:
generating an identifier image of one or both of the medical device and a packaging of the medical device using an optical identifier camera;
parsing the identifier image to extract one or more identifier markers;
analyzing the one or more identifier markers to determine an identity of the medical device;
querying the medical device identity with one or more relational databases to retrieve a predetermined medical imaging parameter;
imaging a target location using a probe of a medical imaging system to generate a medical image;
modifying a medical imaging parameter of the medical imaging system to the predetermined medical imaging parameter; and
placing the medical device at the target location.

24. The method according to claim 23, wherein parsing the identifier image to extract the one or more identifier markers further includes parsing the identifier image into pixels and assigning a numerical value to each pixel and analyzing the numerical values to detect one or more edges within the identifier image.

25. The method according to claim 24, wherein parsing the identifier image to extract the one or more identifier markers further includes analyzing the one or more edges detected within the identifier image to detect one or more boundaries within the identifier image.

26. The method according to claim 23, wherein the one or more identifier markers includes one or more of a boundary of the medical device, a boundary of the packaging, a boundary of a portion of the medical device, a boundary of a portion of the packaging.

27. The method according to claim 25, wherein analyzing the one or more identifier markers to determine the identity of the medical device further includes comparing one or more of a shape and a dimension of the one or more boundaries within the identifier image with a labeled dataset including a plurality of boundaries and associated medical device identities.

28. The method according to claim 23, wherein parsing the identifier image to extract the one or more identifier markers further includes parsing the identifier image into pixels an assigning a numerical value to each pixel and analyzing the numerical values to detect one or more colors within the identifier image.

29. The method according to claim 28, wherein analyzing the one or more identifier markers to determine the identity of the medical device further includes comparing the one or more colors within the identifier image with a labeled dataset including a plurality of colors and associated medical device identities.

30. The method according to claim 24, wherein parsing the identifier image to extract the one or more identifier markers further includes analyzing the one or more edges detected within the identifier image to detect one or more alphanumeric symbols within the identifier image.

31. The method according to claim 30, wherein analyzing the one or more identifier markers to determine the identity of the medical device further includes running a text recognition sub-logic module including comparing the one or more alphanumeric symbols within the identifier image with a labeled dataset including a plurality of alphanumeric symbols associated with medical device identities.

32. The method according to claim 23, wherein the identity of the medical device includes one or more of a model, batch number, serial number, product code, product name, and product description.

33. The method according to claim 23, wherein the medical imaging system is one of an ultrasound medical imaging system, electromagnetic medical imaging system, fluoroscopic imaging system, or a magnetic imaging system.

34. The method according to claim 23, wherein modifying a parameter of the medical imaging system further includes modifying an icon superimposed on the medical image of the target location.
